(19) United States
(12) Patent Application Publication
(10) Pub. No.: US 2011/0118617 A1
(43) Pub. Date: May 19, 2011

(54) Heart Rate Measuring Device and Method for Measuring Heart Rate Thereof

(75) Inventors: Bo-Jau KUO, Taipei (TW); Ching-Hsiu YANG, Taipei (TW)

(73) Assignee: NATIONAL YANG MING UNIVERSITY, Taipei (TW)

(21) Appl. No.: 12/626,376

(22) Filed: Nov. 25, 2009

(30) Foreign Application Priority Data
Nov. 17, 2009 (TW) 098139004

(51) Int. Cl. A61B 5/0404; A61B 5/0468; A61B 5/024

(52) U.S. Cl. 600/515; 600/519

(57) ABSTRACT

This invention discloses a heart rate measuring device and a method for measuring heart rate thereof. The heart rate measuring device can be put on a user body. The heart rate measuring device includes a first electrode, a second electrode, a plurality of optical indicating units, a signal processing circuit and a control unit. When the user's both hands touch the first electrode and the second electrode, the control unit provides a heart signal by using the first electrode, the second electrode and the signal processing circuit. The control unit controls the optical indicating units to output electrocardiographic signals.

### BACKGROUND OF THE INVENTION

1. Field of the Invention

This invention relates to a heart rate measuring device; particularly, this invention relates to a heart rate measuring device indicating heart rate using audio and optical indicating units and the method of measuring heart rate thereof.

2. Description of the Prior Art

All current heart rate analysis devices analyze and collect all kinds of signals using customized circuits, along with tailored signal analysis programs. These tailor-made circuits consist of extremely complicated amplification circuits, and the analysis programs are also created by expensive PC or work stations. Moreover, these analysis programs are all costly tailor-made programs.

The peripheral hardware often costs more than ten thousands dollars, and the computers also costs more than twenty thousand dollars. The price of software can range from two thousands dollars to a million dollars. Apart from the production cost, the technical threshold is also very high, which results in expensive costs and seriously affects and limits the promotion and the application of this technique, such that the population that can utilize the technique is minority.

Moreover, the outputs of the current heart rate measuring devices are often electrocardiographic signals; however, most users, particularly elderly, can not work out the meanings from the electrocardiographic signals. Therefore, current heart rate measuring devices are still not convenient to users.

### SUMMARY OF THE INVENTION

An object of this invention is to provide a heart rate measuring device and a heart rate measuring method thereof, in order to improve the flaws of current techniques.

According to a characteristic of the present invention, the heart rate measuring device proposed by the invention is worn by a user. The heart rate measuring device mentioned above includes a first electrode, a second electrode, a plurality of optical indicating units, signal processing circuits and a control unit. The signal processing circuit above is coupled to the first electrode and the second electrode. The control unit above is coupled to the signal processing circuits and optical indicating units. When both the first electrode and the second electrode are touched by a user, the control unit provides electrocardiographic signals by using the first electrode, the second electrode and the signal processing circuits. The control unit further controls the optical indicating units to output electrocardiographic signals.

According to another characteristic of the present invention, the present invention discloses a method of measuring heart rate, used to measure users' heart rates. The heart rate measuring method includes providing a first electrode and a second electrode; providing a plurality of optical indicating units; when the first electrode and the second electrode, the process of measuring heart rate starts and electrocardiographic signals are generated; and the optical indicating units are controlled to output electrocardiographic signals.

The advantage of this invention is that heart rate measuring device can be used at anytime of a day and the heart rates are indicated using optical indicating units (e.g. LED) with different colors, or audio indicating units. Moreover, any abnormal conditions are specially indicated. With this invention, the subjects that are overstrain or seriously sick can remind themselves. The users can know if they need to change their life styles or be further examined by doctors.

In order to clarify the above invention and other objects, characteristics and advantages, more detailed description will be given below with reference to the appended drawings.

### BRIEF DESCRIPTION OF THE DRAWINGS

FIG. 1A schematically illustrates the heart rate measuring device in accordance with embodiments of the present invention worn by a subject.

FIG. 1B schematically shows the shape the heart rate measuring device in accordance with embodiments of the present invention.

FIG. 2 schematically illustrates the block diagram showing the functions of the components of the heart rate measuring device in accordance with embodiments of the present invention.

FIG. 3 illustrates a flow chart of the heart rate measuring method in accordance with embodiments of the present invention.

### DETAILED DESCRIPTION OF THE INVENTION

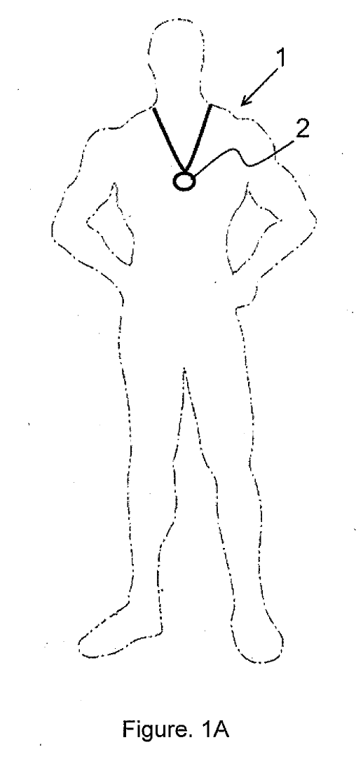

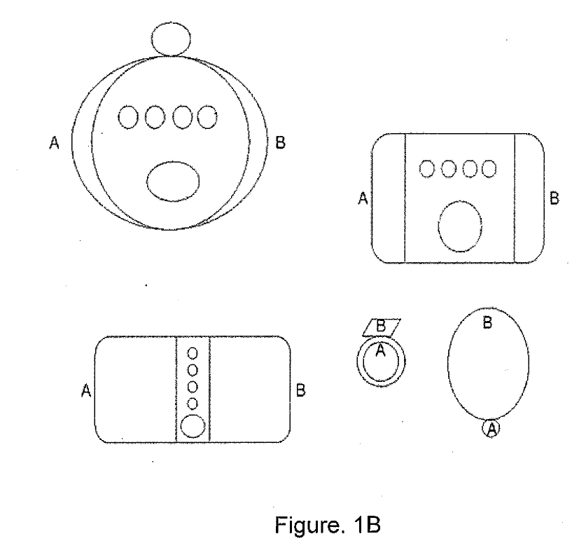

FIG. 1A schematically illustrates the heart rate measuring device in accordance with embodiments of the present invention worn by a subject. The heart rate measuring device 2 provided by the preferred embodiment of this invention can be put on a user in the form of a necklace. As illustrated by FIG. 1B, the shape of the heart rate measuring device 2 provided by the preferred embodiment of this invention can be round, flat round, square, rod (with the size of mobile phone accessories), key ring, or even as small as a finger ring, or necklace pendant; wherein label A and label B can be the electrodes. In this invention, the shape of this invention is not limited, as long as the shape of the heart rate measuring device 2 is convenient for users to carry.

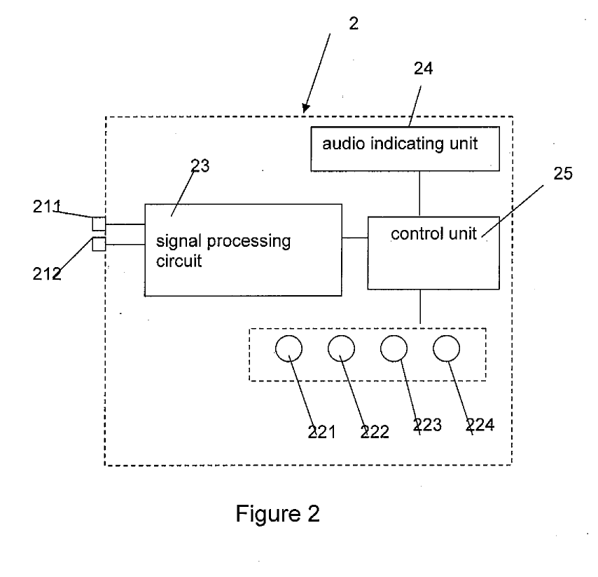

FIG. 2 schematically illustrates the block diagram showing the functions of the components of the heart rate measuring device in accordance with embodiments of the present invention. The embodiment provided by the heart rate measuring device 2 includes the first electrode 211, the second electrode 212, optical indicating units 221, 222, 223, 224, signal processing circuit 23, audio indicating unit 24, and control unit 25.

The signal processing circuit 23 is coupled to the first electrode 211 and the second electrode 212. The control unit 25 is coupled to the signal processing unit 23, optical indicating units 221, 222, 223, 224 and audio indicating unite 24.

In this embodiment, the first electrode 211 and the second electrode 212 are used to detect electrocardiographic signals, and the dry electrodes with a housing having the shape of a rod can be adopted. Moreover, the shapes are not limited. In other embodiments, the first electrode 211 and the second electrode 212 may also adopt the conventional electrocardiographic electrode or sensor input electrode and any other electrodes that can gain electrocardiography or heart rate information. However, this invention is not limited to the aforementioned means.

In this embodiment, the signal processing circuit 23 is used to process the electrocardiographic signals gained by the first electrode 211 and the second electrode 212. In this embodiment, the signal processing circuit 23 includes filter circuit, signal amplification circuit, analog-to-digit converter (ADC) and digital signal processor, wherein the analog amplifier of the signal amplification circuit can be a difference amplifier or a single-point digital amplifier.

In this embodiment, the optical indicating units 221, 222, 223, 224 can be LEDs with different colors, for example, the optical indicating unit 221 can be blue LED, the optical indicating unit 222 can be green LED, the optical indicating unit 223 can be yellow LED, and the optical indicating unit 224 can be red LED. In other embodiments, other luminescent objects can also be used as the optical indicating units 221, 222, 223, 224, and this invention does not further limit the use of the luminescent objects.

In this embodiment, when the four optical indicating units 221, 222, 223, 224 are all on or flashing, it is indicated that a user is detected with cardiac arrhythmia. If the optical indicating unit 221 is on, it is indicated that the heart rate is less than 60 per minute (too slow); If the optical indicating unit 222 is on, it is indicated that the heart rate is between 60 and 80 per minute (normal); If the optical indicating unit 223 is on, it is indicated that the heart rate is between 80 and 100 per minute (a little fast); If the optical indicating unit 224 is on, it is indicated that the heart rate is more than 100 per minute (too fast).

Moreover, when the optical indicating unit 221 or the optical indicating unit 224 is on, the control unit 25 can also control the audio indicating unit 24 to make a sound to remind users.

Moreover, the audio indicating unit 24 can also be controlled by the control unit 25, so as to make a sound with the electrocardiographic signals of each heart beat. When the heart rate reaches a preset range (e.g. too fast or too slow), the control unit 25 can even make the audio indicating unit 24 make an alarm sound to warn a user to pay attention.

In this embodiment, the control 25 can analyze the detected electrocardiographic signals, and further analyze the periods of heart rates. Moreover, the control unit 2 can control the operation of the optical indicating units 221, 222, 223, 224 and audio indicating unit 24.

In this embodiment, when the first electrode 211 and the second electrode 212 are both touched by a user (e.g. the first electrode 211 is touched by the right hand, and the second electrode 212 is touched by the left hand), the control unit 25 can turn on the heart rate measuring device 2 automatically; when the user's hands are not both in contact with the first electrode 211 and the second electrode 212, the control unit 25 can turn off the heart rate measuring device 2 automatically to conserve energy.

As mentioned above, when the first electrode 211 and the second electrode 212 are both touched by the user, the first electrode 211 and the second electrode 212 detect electrocardiographic signals and output the electrocardiographic signals to signal processing circuit 23 to process the signals. After that, the results are transmitted to the control unit 25, so that the control unit 25 can provide electrocardiographic signals. The control unit 25 can control optical indicating units 221, 222, 223, 224 according to the electrocardiographic signals, to indicate the corresponding status settings of the electrocardiographic signals.

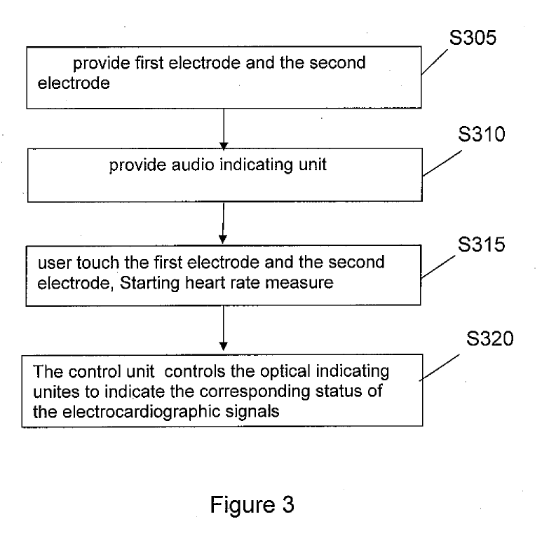

FIG. 3 illustrates a flow chart of the heart rate measuring method in accordance with embodiments of the present invention. The description of FIG. 3 can be referred to that of FIG. 2. Because most operation of this invention has been disclosed above, the description of the operation below will only be lightly touched on.

In step S305, the first electrode 211 and the second electrode 212 are provided.

In step S310, the optical indicating units 221, 222, 223, 224 are provided.

In step S315, when the first electrode 211 and the second electrode 212 are both touched by a user, the heart rate measuring device 2 is turned on automatically and starts measuring the heart rate of the user to further generate electrocardiographic signals; and in step S320, the control unit 25 controls the optical indicating units 221, 222, 223, 224 to indicate the corresponding status of the electrocardiographic signals.

As mentioned above, the heart rate measuring device provided by this invention is not only cheap but also portable. This invention not only can be used as a key ring or a decoration, but also used to measure heart rate and give feedback on the improvement of heart beat status. With the two aforementioned functions, the applications of electrocardiographic signals can be efficiently promoted in the fields of medication, research and daily life.

Although this invention has been disclosed better as above by the preferred embodiments, they are not intended to limit the scope of the present invention. An ordinary skilled person in the art can make any modifications and improvements without departing from the spirit and the scope of this invention. Therefore, the protection scope of this invention is defined by the appended claims.

What is claimed is:

1. A heart rate measuring device, worn by a subject, comprising:
a first electrode;
a second electrode;
a plurality of optical indicating units;
a signal processing circuit, coupled to the first electrode and the second electrode; and
a control unit, coupled to the signal processing circuit and the optical indicating units, wherein when the first electrode and the second electrode are both in contact with the subject, the control unit provides electrocardiographic signals by using the first electrode, the second electrode and the signal processing circuit, the control unit further controlling the optical indicating units to output electrocardiographic signals.

2. The heart rate measuring device of claim 1, wherein the first electrode and the second electrode are used to detect the electrocardiographic signals.

3. The heart rate measuring device of claim 1, wherein the first electrode and the second electrode are used to detect electrocardiographic signals, the signal processing unit used to process the detected electrocardiographic signals.

4. The heart rate measuring device of claim 1, wherein the heart rate measuring device is off when the first electrode and the second electrode are not both in contact with both hands of the subject.

5. The heart rate measuring device of claim 1, wherein the optical indicating units include a first optical indicating unit, a second optical indicating unit, a third optical indicating unit and a fourth optical indicating unit, indicating that the subject is detected with cardiac arrhythmia as all four lights are on or flashing.

6. The heart rate measuring device of claim 1, wherein the optical indicating units include a first optical indicating unit, a second optical indicating unit, a third optical indicating unit and a fourth optical indicating unit, indicating that the heart rate of the subject is too slow as the first optical indicating unit is on.

7. The heart rate measuring device of claim 6, wherein when the second optical indicating unit is on, the subject's heart rate is normal.

8. The heart rate measuring device of claim 6, wherein when the third optical indicating unit is on, the subject's heart rate is a little fast.

9. The heart rate measuring device of claim 6, wherein when the third optical indicating unit is on, the subject's heart rate is too fast.

10. The heart rate measuring device of claim 1, further including an audio indicating unit coupled to the control unit.

11. The heart rate measuring device of claim 10, wherein the audio indicating unit makes a sound with each heart beat along the electrocardiographic signals.

12. A heart rate measuring method, used to measure a subject's heart rate, comprising:
providing a first electrode and a second electrode;
providing a plurality of optical indicating units;
when the first electrode and the second electrode are both in contact with the subject, the device starts measuring the heart rate of the subject to generate electrocardiographic signals; and
controlling the optical indicating units to output the electrocardiographic signals.

13. The heart rate measuring method of claim 12, wherein the first electrode and the second electrode are both used to detect the electrocardiographic signals.

14. The heart rate measuring method of claim 12, wherein the heart rate measuring progression is stopped when the first electrode and the second electrode are not both in contact with the subject's hands.

15. The heart rate measuring method of claim 12, wherein the optical indicating units include a first optical indicating unit, a second optical indicating unit, a third optical indicating unit, and a fourth optical indicating unit, when all the optical indicating units are on or flashing, indicating that the subject is detected with cardiac arrhythmia as all four lights are on or flashing.

16. The heart rate measuring method of claim 12, wherein the optical indicating units include a first optical indicating unit, a second optical indicating unit, a third optical indicating unit and a fourth optical indicating unit, indicating that the heart rate of the subject is too slow as the first optical indicating unit is on.

17. The heart rate measuring method of claim 16, wherein when the second optical indicating unit is on, the subject's heart rate is normal.

18. The heart rate measuring method of claim 16, wherein when the third optical indicating unit is on, the subject's heart rate is a little fast.

19. The heart rate measuring method of claim 16, wherein when the third optical indicating unit is on, the subject's heart rate is too fast.

20. The heart rate measuring method of claim 16, further including an audio indicating unit coupled to the control unit, the audio indicating unit making a sound with each heart beat along the electrocardiographic signals.
